(19) United States
(12) Patent Application Publication
(10) Pub. No.: US 2016/0334371 A1
(43) Pub. Date: Nov. 17, 2016

(54) METHOD AND DEVICE FOR DETERMINING THE PROPORTION OF MOLECULAR OXYGEN IN A RESPIRATORY GAS BY MEANS OF SOUND

(71) Applicant: PETER GANSHORN, Münnerstadt (DE)

(72) Inventor: PETER GANSHORN, Münnerstadt (DE)

(21) Appl. No.: 15/112,322

(22) PCT Filed: Jul. 31, 2015
(86) PCT No.: PCT/DE2015/100323

§ 371 Date: Jul. 18, 2016

(30) Foreign Application Priority Data
Aug. 8, 2014 (DE) 10 2014 111 366.7

(51) Int. Cl. G01N 29/024; A61B 5/083; A61B 5/097; G01N 33/497

(52) U.S. Cl. CPC G01N 29/024; G01N 33/497; A61B 5/0836; A61B 5/0833; A61B 5/097; A61B 2562/0204; A61B 2010/0087

(57) ABSTRACT

The invention relates to a method for determining the proportion of molecular oxygen in a respiratory gas, for example in lung function diagnostics, comprising the introduction of the respiratory gas into a measurement tube, transmitting a sound signal by means of a sound transmitter and receiving the sound signal by means of a sound receiver, defining a sound measurement zone by means of the sound transmitter and the sound receiver, determining the average molar mass of the respiratory gas by means of a sound propagation time measured over the measurement zone, and determination of the carbon dioxide content of the respiratory gas with a carbon dioxide gas sensor. The invention also relates to a device for performing the method.

The invention relates to a method for determining the proportion of molecular oxygen in a respiratory gas, for example in lung function diagnostics, comprising the introduction of the respiratory gas into a measurement tube, transmitting a sound signal by means of a sound transmitter and receiving the sound signal by means of a sound receiver, defining a sound measurement zone by means of the sound transmitter and the sound receiver, determining the average molar mass of the respiratory gas by means of a sound propagation time measured over the measurement zone, and determination of the carbon dioxide content of the respiratory gas with a carbon dioxide gas sensor. The invention also relates to a device for performing the method.

In a large number of medical applications, the determination of the proportion of molecular oxygen in the respiratory gases is essential, for example in emergency care and intensive care, anaesthesia and lung function diagnostics. By means of the determined oxygen proportion, conclusions can be drawn about the lung functionality, the respiratory quotients and the metabolic rate of the test subject.

Conventional systems for studying respiratory gases contain a large number of different sensors and are based on the measurement of various respiratory gas parameters, such as flow rate, temperature, humidity, pressure and the specific respiratory gas composition. Determination of the composition of the respiratory gas is performed predominantly in the side-stream or main-stream with the aid of different, in some cases more complicated and more expensive measurement methods, such as, for example, infrared spectroscopy, paramagnetism, laser or mass spectroscopy.

Respiratory gas typically consists of the components or elements mentioned below: Nitrogen (N₂), oxygen (O₂), argon (Ar), water vapour (H₂O) and various trace gases such as carbon dioxide (CO₂), ozone, carbon monoxide and various noble gases. The average molar mass of the respiratory gas results from the total of the products of the molar masses and amount contents of the individual components, principally, however, from the total of the products of the molar masses of oxygen, nitrogen, carbon dioxide and argon.

The average molar mass of the respiratory gas, however, is also dependent on its humidity, and therefore on pressure and temperature. If the respiratory gas, for example, contains moisture, its average molar mass is reduced, since the molar mass of water vapour is smaller than the average molar mass of dry respiratory gas. This dependence of the average molar mass of the respiratory gas leads the fact that, to determine the composition of the respiratory gas, in many cases complicated and expensive measurement methods, along with complicated computations are necessary.

Modern methods and devices are based on determining the average molar mass of a gas mixture by ultrasound measurement. Such a method is disclosed, for example, in the application document (Offenlegungsschrift) EP 0 533 980 A1, in which the concentration of fuels or gases are determined in the intake air of vehicle engines. For medical applications, devices for measuring the molar mass of gas mixtures by means of ultrasound propagation measurement are known. For example, application document (Offenlegungsschrift) EP 1 279368 A2 describes a device for measuring the flow velocity and/or the molar mass of gases and gas mixtures in medical application by means of ultrasound propagation measurement.

For example, application document (Offenlegungsschrift) EP 0 646 346 A2 describes a device for measuring respiratory gas parameters comprising a respiratory tube, ultrasound sensors and preamplifier electronics disposed in a separate housing.

By means of ultrasound measurement, the density of the respiratory gas can be measured in order to determine therefrom the average molar mass of the respiratory gas, however additional information is necessary, such as, for example, its temperature, its humidity, its pressure and/or its velocity.

Using the above-described methods, however, no direct conclusions can be drawn from the average molar mass of the respiratory gas about its specific composition or the concentration of the individual components or elements, such as, for example, the proportion of molecular oxygen in the respiratory gas.

Patent application EP 0 653 919 B1 describes a method for measuring the molar mass of gases or gas mixtures by means of ultrasound measurement and a device for performing this method. In addition, it is disclosed that, due to the combination of ultrasound measurement with a further gas sensor, the oxygen uptake, the carbon dioxide emission and the respiratory quotient can be determined. The patent document also describes an infrared-based carbon dioxide gas sensor.

Though the systems available in the prior art are capable of determining a large number of different respiratory gas parameters, this is performed with a comparatively high technical outlay of this partly complex and therefore fault-prone method and also devices. For most medical systems, however, only the proportion of molecular oxygen and carbon dioxide in the respiratory gas is of central importance. There is therefore a huge need for a method that, in a simple and reliable manner, only determines the proportion of molecular oxygen in respiratory gases.

The object of the invention is thus to provide a simple and reliably operating method for determining the proportion of molecular oxygen in respiratory gases, which performs the determination in an extremely short time in order to overcome the above-described difficulties. Another object of the invention is to obtain a small and compact device for performing the method.

This object is achieved by means of the method with the features of the independent claim, in particular in that the determination of the proportion of molecular oxygen in the respiratory gas is performed from the determined average molar mass of the respiratory gas and from the determined carbon dioxide proportion. Advantage embodiments of the invention that are realised individually or in combination are described in the subclaims.

The term “respiratory gas” describes that gas mixture that can be used for respiration, such as, for example, ambient air or various gas mixtures that are used in respiratory devices.

The term “measurement tube” is known to the person skilled in the art and concerns any hollow body which is suitable for allowing respiratory gas to be introduced. The measurement tube is a bore in a block, consisting of an arbitrary material, preferably of plastic, metal or a ceramic material, such as, for example, a metal block or is a tube consisting of an arbitrary material, preferably of plastic, metal or a ceramic material. The used measurement tube is preferably easy to clean and/or has a smooth surface, that is to say the used material is at least acid and/or alkali resistant. Alternatively, the measurement tube is gastight. The measurement tube comprises a sound transmitter and a sound receiver as sound measurement zone, and a carbon dioxide gas sensor. In addition, in order to increase the accuracy of determining the proportion of molecular oxygen, the measurement tube can include a device for measuring temperature, air pressure and/or the humidity of the respiratory gas.

The term “sound transmitter” is known and is a source or device for generating and radiating sound waves. Corresponding to the frequency range, a person skilled in the art distinguishes between infrasound (<16 Hz), audible sound (16 Hz to 20 kHz), ultrasound (20 kHz to 1.6 GHz) and hypersound (>1 GHz). Preferably ultrasound is used, so that the sound transmitter is at least one ultrasound source.

The term “sound receiver” is known and is an acoustic sensor or a microphone that converts airborne sound as cyclic sound pressure oscillations into corresponding tension changes, preferably into electrical voltage changes. Preferably ultrasound is used, so that the sound receiver is at least one acoustic sensor or a microphone. A person skilled in the art is familiar with the construction and mode of operation of a sound transmitter and a sound receiver.

In one embodiment, ultrasound is preferably used in the present invention. In such a case, it is expedient to use piezo oscillators as sound transmitters and sound receivers. Even more preferred is to operate one and the same piezo oscillator both for the determination of the average molar mass of the respiratory gas alternately as a sound transmitter and as a sound receiver.

Preferably, the sound transmitter and the sound receiver are undetachably integrated onto or into the measurement tube, so that they form a single part with the measurement tube. They are then cast for example into corresponding shoulders of the measurement tube. In this manner, the measurement tube with the sound transmitter and sound receiver can be readily exchanged. Preferably, the sound transmitter and the sound receiver are undetachably connected to the measurement tube, so that they and the measurement tube can be easily separated from one another. In this manner, the measurement tube with the sound transmitter and sound receiver can be exchanged independently of one another.

Furthermore, the sound transmitter and/or the sound receiver preferably comprises preamplifier electronics, which are undetachably integrated into or detachably connected to the measurement tube. A person skilled in the art knows various possibilities for realising this.

The sound transmitter and the sound receiver define a sound measurement zone of known length, which preferably extends within or through the measurement tube. The sound transmitter and the sound receiver can here assume any position with respect to one another, for example opposite one another or side by side. It goes without saying that, for example in the case of them lying side by side, that end of the measurement tube that is opposite the sound transmitter is formed such that the wave generated and emitted by the sound transmitter is reflected and is subsequently received by the sound transmitter. This is achieved, for example, by the use of a reflection surface. Preferably, the sound transmitter and the sound receiver are arranged such that they are opposite one another. Even more preferred, the sound transmitter and the sound receiver are arranged such that the sound measurement zone extends along the flow direction of the respiratory gas in the measurement tube. This can take place directly, for example parallel to the flow direction, or else indirectly, for example with single or repeated crossing of the flow of the respiratory gas. The sound measurement zone passes a total of two times in the opposite direction, that is to say once in the direction of the flow and once in the counter direction in order to eliminate the influence of the flow velocity on the measured sound velocity. Overall, a twofold passage of the sound measurement zone thereby results, so that as a result the influence caused by superimposition of the flow velocity is averaged out. This has the result that the sound measurement zone can only pass through an even number of times, that is to say it can pass through preferably two times, four times, six times, eight times, ten times, twenty times, etc.

In principle, and as can be proven from the physics literature, the sound velocity in gases can be calculated from the formula:

$${C}_{s}^{2} = κ \frac{p}{ρ}$$

where κ represents the adiabatic coefficient, which is determined from the quotient of the specific heat cₚ and cᵥ, and p the pressure and ρ the density. By transformations in which the density is divided by the total mass m and is replaced by the total volume V, and with the application of the ideal gas equation

pV=nRT
one obtains

$${C}_{s}^{2} = κ \frac{RT}{M}$$

where M is the molar mass, m the total mass and n the number of moles. By measuring the corresponding parameters, the molar mass is obtained, where the molar mass serves for identifying the corresponding gases. The total mass, on the other hand, is insignificant.

The relationships just derived apply for the determination of one-component gases. Respiratory gas contains a large number of gas components, so that the measured molar mass represents a value in which the individual gas components are weighted with the corresponding proportions in the gas mixture for forming an average value of the molar mass. If the proportions of the individual components in inhaled gas or ambient gas are compared with the exhaled gas, they remain constant, with the exception that the exhaled gas contains a higher CO₂ and a lower O₂ proportion than the inhaled or ambient gas.

Over this sound measurement zone, the sound propagation time is measured, preferably by electronic means. From the sound propagation time, the flow rate of the respiratory gas and/or the average molar mass of the respiratory gas can be determined. The flow velocity (c) is preferably calculated by means of the known formula:

$$c = a * \frac{{t}_{1} - {t}_{2}}{{t}_{1} * {t}_{2}}$$

where c is the flow velocity, a is a dimensional constant and t₁ and t₂ are sound propagation times measured over the sound measurement zone.

The sound propagation time is a characteristic that, depending on the ambient parameters, is proportional to the mean molar mass of the respiratory gas. The average molar mass of the respiratory gas (M_{respiratory gas}) is calculated with the aid of the formula:

$$Mrespiratory gas = b * {T ( \frac{{t}_{1} * {t}_{2}}{{t}_{1} + {t}_{2}} )}^{2}$$

where M_{respiratory gas} is the mean molar mass of the respiratory gas, T is the determined temperature of the respiratory gas, b is a dimensional constant and t₁ and t₂ are the sound propagation times measured over the sound measurement zone.

Within the scope of the invention, the determination of the carbon dioxide proportion of the respiratory gas can be performed with an arbitrary carbon dioxide gas sensor. However, it is conventional to determine using infrared, that is to say for the determination an infrared transmitter and an infrared receiver are necessary. The carbon dioxide sensor is therefore preferably an infrared receiver that receives an infrared signal in the carbon dioxide absorption band transmitted by an infrared transmitter. The carbon dioxide proportion in the respiratory gas is then determined from the infrared signal received via the infrared measurement zone.

The term “infrared transmitter” is known and is a source or device for generating and radiating electromagnetic waves in the spectral range between visible light and the longer wave terahertz radiation (1 mm and 780 nm).

The same applies for the term “infrared receiver”, which is an optical sensor for the aforementioned wavelength range. A person skilled in the art is familiar with the construction and mode of operation of an infrared transmitter and an infrared receiver.

Preferably, the infrared transmitter and the infrared receiver are undetachably integrated onto or into the measurement tube, so that they form a single part with the measurement tube. They are then cast for example into corresponding shoulders of the measurement tube. In this manner, the measurement tube with the infrared transmitter and infrared receiver can be readily exchanged. Preferably, the infrared transmitter and the infrared receiver are undetachably connected to the measurement tube, so that they and the measurement tube can be easily separated from one another. In this manner, the measurement tube with the infrared transmitter and infrared receiver can be exchanged independently of one another.

Furthermore, the infrared transmitter and the infrared receiver preferably comprises preamplifier electronics, which are undetachably integrated into or detachably connected to the measurement tube. A person skilled in the art knows various possibilities for realising this.

The infrared transmitter and infrared receiver define an infrared measurement zone of known length, which preferably passes through the measurement tube. The infrared transmitter and the infrared receiver can assume any position with respect to one another, for example opposite one another and/or side by side with one another. It goes without saying that, for example in the case of them lying side by side, that end of the measurement tube that is opposite the infrared transmitter is formed such that the light generated and emitted by the infrared transmitter is reflected and is subsequently received by the infrared transmitter. This is achieved, for example, by the use of a mirror. Preferably, the infrared transmitter and the infrared receiver are arranged such that they are opposite one another.

Even more preferred, the infrared transmitter and the infrared receiver are arranged such that the infrared measurement zone crosses the flow direction of the respiratory gas in the measurement tube. In an arrangement outside the measurement tube, at least one optically pervious range is provided at a corresponding place for passage of the infrared signal. The optically pervious region is preferably a crystal window. It is necessary that the crystal window in or on the measurement tube closes the latter with a gas-tight seal.

The determination of the carbon dioxide content in the respiratory gas is based on the fact that carbon dioxide molecules absorb the incident infrared light waves, whose frequency lies in the absorption spectrum of carbon dioxide. For the measurement, an infrared transmitter and an infrared receiver are mounted opposite one another across an infrared measurement zone. At the same time, the infrared transmitter and infrared receiver define the infrared measurement zone. The light waves of the infrared transmitter excite the carbon dioxide molecules to oscillations. The carbon dioxide molecule returns with a time delay to its unexcited original state and emits the absorbed energy, in the form of concentric radiation of infrared light again. Since only a small part of the infrared light emitted by the carbon dioxide molecule propagates in the same direction as the infrared light of the infrared transmitter, in the presence of carbon dioxide molecules in the infrared measurement zone the intensity of the infrared light that is incident on the infrared receiver is measurably attenuated. Although scattering effects of aerosols and air molecules occur, they are negligible for the measurement.

The measurement of the attenuation of the intensity of the infrared signal or infrared light emitted by the infrared transmitter is thus in direction proportional relationship to the quantity of carbon dioxide molecules within the infrared measurement zone. That is to say the larger the proportion of carbon dioxide in the respiratory gas, the less infrared radiation reaches the infrared receiver. The smaller the proportion of carbon dioxide in the respiratory gas, the more infrared radiation reaches the infrared receiver. If, on the other hand, there are no carbon dioxide molecules in the respiratory gas, then the infrared ray transmitted by the infrared transmitter passes completely to the infrared receiver.

The term “means for measuring the temperature of the respiratory gas” concerns a measurement device for determining the temperature, for example a thermometer or thermocouple. Within the scope of the present invention, the temperature can also be electrically measured.

Such a means is undetachably integrated into the measurement tube or detachably connected thereto. A person skilled in the art knows various possibilities for realising this. In addition, preamplifier electronics may be comprised.

The term “means for measuring the air pressure of the respiratory gas” is known to the person skilled in the art and concerns a measurement device for recording and/or for displaying the physical pressure of the respiratory gas. Within the scope of the present invention, any arbitrary pressure sensor can be used, preferably one that convert the physical parameter pressure into an electrical output parameter as a measure of the pressure.

The pressure sensor is undetachably integrated into the measurement tube or detachably connected thereto. A person skilled in the art knows various possibilities for realising this. It may also comprise preamplifier electronics.

The term “means for measuring the humidity of the respiratory gas” concerns a measurement device for determining the atmospheric humidity, for example a hygrometer. Within the scope of the present invention, the temperature can also be electrically measured, for example in that a humidity sensor provides an electrical signal.

Such a means is undetachably integrated into the measurement tube or detachably connected thereto. A person skilled in the art knows various possibilities for realising this. In addition, preamplifier electronics may be comprised.

The term “determination of the proportion of molecular oxygen in the respiratory gases” concerns the determination of the proportion or concentration of molecular oxygen in respiratory gases. The determination is performed indirectly, preferably by forming a difference from the determined mean molar mass of the respiratory gas and from the determined carbon dioxide proportion of the respiratory gas.

The difference formation is based on the fact that the mean molar mass of the respiratory gas and the carbon dioxide content are determined only once, namely in the inhalation gas or in the ambient gas and/or in the exhalation gas. The values measured in the inhalation gas or in the ambient gas can be used for calibrating the method or for zero point adjustment, so that the proportion of molecular oxygen can be determined directly from the difference between the determined mean molar mass of the exhalation gas and the determined carbon dioxide proportion of the exhalation gas.

The values measured in the inhalation gas or in the ambient gas are used for each determination of the proportion of molecular oxygen for calibration of the process or for zero point adjustment, so that the proportion of molecular oxygen in the exhalation gas can be determined directly by difference formation. This means that directly before each determination, the values measured in the inhalation gas or in the ambient gas are used for calibration of the process or for zero point adjustment, before the determination of the proportion of molecular oxygen in the corresponding exhalation gas subsequently takes place.

Alternatively, the values measured in the inhalation gas or in the ambient gas are not used for each determination of the proportion of molecular oxygen for calibration of the process or for zero point adjustment. This means that directly before each determination, the values measured in the inhalation gas or in the ambient gas are used for calibration of the process or for zero point adjustment, before only the determination of the proportion of molecular oxygen in the corresponding exhalation gas subsequently takes place by difference formation and without further calibration or zero point displacement.

The determination by means of difference formation is possible, since the absolute quantities of the individual components or elements of the respiratory gas, such as nitrogen, argon and various trace gases, in the exhalation gas does not change in comparison to the inhalation gas or ambient gas during the measurement. For the process, it is therefore assumed as simplification that their concentrations are constant. The influence of these components or elements of the respiratory gas can therefore be summarized in a dimensional constant in the measurement.

In addition, it is assumed for the method that further influences, such as the flow velocity and humidity of the respiratory gas do not need to be considered for determination of the proportion of molecular oxygen in the exhalation gas for the reasons mentioned below.

In the considerations on the constancy of the values, it is assumed that the value of humidity in the exhalation gas is constant and is approximately 100%. In the normal case, the influence of humidity is therefore negligible. For this assumption, it is preferably assumed that a possible feed line to the measurement tube does not exceed a particular length to avoid significant cooling with displacement of the dew point, and consequently absence of the humidity. In addition, however, cases can occur in which a humidity measurement is appropriate, for example with a long line to the measurement tube and/or a large temperature difference between the inhalation or ambient air and the exhalation gas, so that because of significant cooling a displacement of the dew point and consequently an absence of the humidity occurs. The influence of the flow velocity is eliminated in that the sound measurement zone is, overall, passed through twice in opposite directions.

There is thus a direct relationship between the proportion of molecular oxygen on one hand and the difference between the average molar mass of the respiratory gas and the concentration of carbon dioxide on the other hand. This advantageously has the result that the determination of the proportion of molecular oxygen takes place in an extremely short time and without complicated calculation. The determination of the proportion of molecular oxygen preferably takes place by means of known mathematical procedures or, particularly preferred, from the empirically determined dependency of the difference of the measured electrical signals. The proportion of molecular oxygen (C_{M O}_{₂}) is preferably calculated by means of the following formula:

C_{M O}_{₂}=k₁(M_{respiratory gas}k₂*C_{M CO}_{₂}−k₃)

where C_{M O}_{₂} is the proportion of molecular oxygen in the respiratory gas M_{respiratory gas} is the average molar mass of the respiratory gas, C_{M CO}_{₂} is the proportion of carbon dioxide in the respiratory gas and k₁, k₂ and k₃ represent dimensional constants.

The measurement of the inhalation gas or of the ambient gas takes place immediately before measurement of the respiratory gas.

For increasing the accuracy of determination of the proportion of molecular oxygen, the temperature and/or the humidity of the respiratory gas is additionally measured. Even more preferred, the temperature of the respiratory gas is measured. The measurement is performed by means of the above-described measurement devices.

Because of this additional measurement or measurements, it is possible to increase the accuracy of the determination and also to perform a control of the determination. For example, a control of the determination may be appropriate if the temperature and/or the humidity of the inhalation gas or of the ambient gas is different from that of the exhalation gas. For example with a long feed line to the measurement tube, a control of the determination may be appropriate.

It is assumed that the definitions and explanations of the aforementioned terms apply for all the aspects described in this description, unless otherwise stated.

The invention further concerns a device for determination of the proportion of molecular oxygen in a respiratory gas and proposes, to achieve the object, that the device is characterised by an evaluation unit for determination of the proportion of molecular oxygen, the evaluation unit determining the difference from the determined average molar mass of the respiratory gas and from the determined carbon dioxide proportion. For this purpose, the two measured signals are fed to a subtraction element for difference formation. Advantage embodiments of the invention that are realised individually or in combination are described in the subclaims.

The device is preferably designed so as to be gastight. The gastight design of the device also extends to all the components or elements comprised by the device.

Alternatively, the measurement tube is connected in a gastight manner to the device. The measurement tube is particularly preferably connected in an undetachable or detachable manner to the device. In the case of a detachable connection, the measurement tube can be exchanged.

The device can alternatively be designed as a block, which may consist of any arbitrary material, for example of metal. The device, as a measurement tube, comprises a bore. In this case, the components or elements comprised by the measurement tube are integrated in the device.

The term “evaluation unit” concerns an auxiliary means, for example a computer or a memory module, which is capable of recording, analysing, processing, storing and/or transmitting the particular measurement values or the measured signals. The input is received by the evaluation unit via the transmitters, receivers, means for measurement comprised by the device and/or via a comprised computation unit or else via input by human users. Preferably the evaluation unit is a computer or a digital memory module, for example a flash memory, or an electronic memory module, for example an erasable programmable read-only memory (EPROM) or an electrically erasable programmable read-only memory (EEPROM).

The evaluation unit alternatively indirectly determines the proportion of molecular oxygen in the respiratory gas by subtraction of the electrical measurement signals. The subtraction element only determines the difference between the determined measurement values or the measured electrical signals, that is to say the difference from the determined average molar mass of the respiratory gas and from the determined carbon dioxide proportion of the respiratory gas. The subtraction element preferably determines the difference by means of the following formula:

C_{M O}_{₂}=k₁(M_{respiratory gas}−k₂*C_{M CO}_{₂}−k₃)

where C_{M O}_{₂} is the proportion of molecular oxygen in the respiratory gas M_{respiratory gas} is the average molar mass of the respiratory gas, C_{M CO2} is the proportion of carbon dioxide in the respiratory gas and k₁, k₂ and k₃ represent dimensional constants.

The determination of the proportion of molecular oxygen preferably takes place from the empirically determined dependency of the determined difference, particularly preferably from the empirically determined dependency of the determined difference of the measured electrical signals.

Preferably, for increasing the accuracy of the determination of the proportion of molecular oxygen, the device additionally comprises a device for measuring the temperature, the air pressure and/or the humidity of the respiratory gas. Even more preferably, a device for measuring the temperature and/or the air pressure of the respiratory gas. These devices are also suitable for a control of this determination.

Alternatively the device comprises at least one connection for feeding and removing the respiratory gas, wherein this is mounted on the measurement tube. Even more preferably, the device comprises two connections for supplying and removing the respiratory gas. Particularly preferably the connections are mounted on the mutually opposite ends of the measurement tube, even more preferably they are mounted so as to be perpendicular to the axis of the measurement tube, wherein they can assume any desired angle with respect to one another. Preferably, the device comprises two different connections for supplying and removing the respiratory gas. Particularly preferably the respiratory gas that is introduced via the connection for supply flows through the measurement tube and emerges again from the connection for removal.

Further details, features and advantages of the invention can be taken from the following description of the preferred embodiment in conjunction with the subclaims.

Herein, the respective features can be realized independently or severally in combination with one another. The invention is not limited to the exemplary embodiment. The exemplary embodiment is illustrated diagrammatically in the figures. The same reference numerals in the individual figures designate elements that are the same or functionally the same, or which correspond to one another as regards their function.

In detail, the figures show:

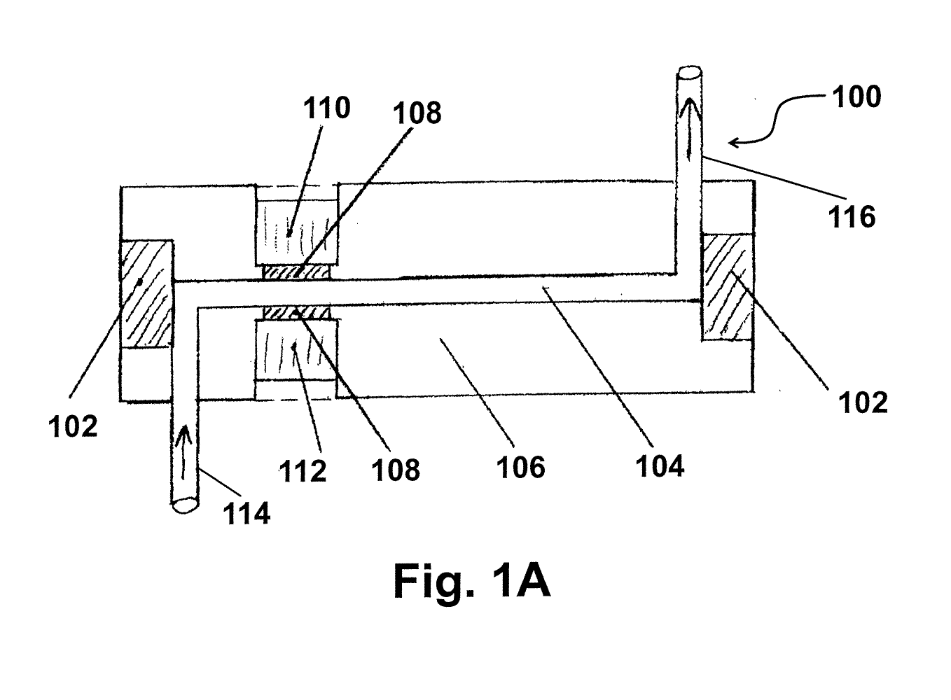

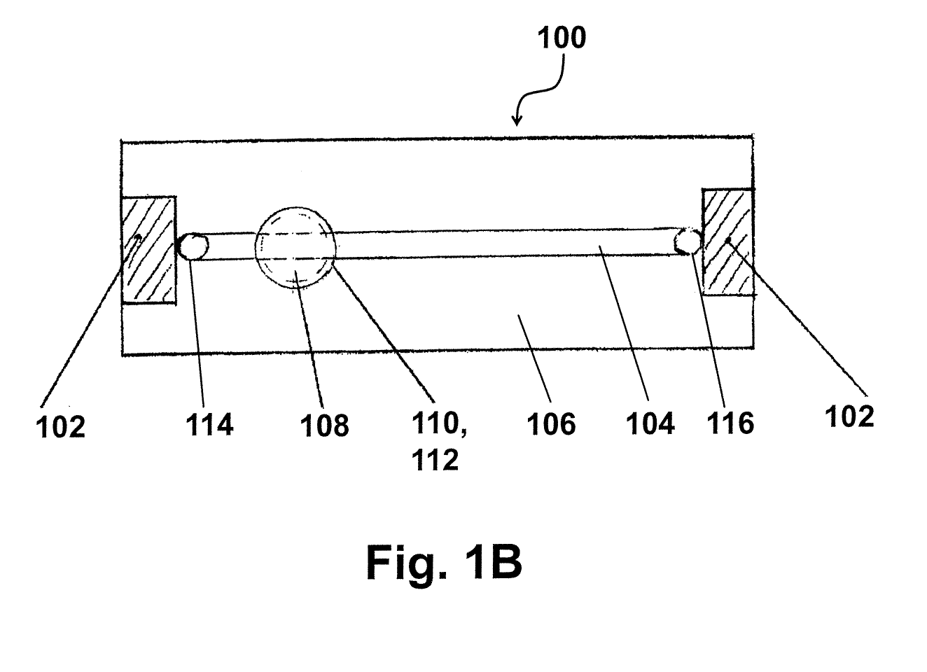

FIGS. 1A-1B shows the device according to the invention in different views.

In FIGS. 1A to 1B, a device according to the invention (100) is shown in schematic view in top view (FIG. 1A) and in front view (FIG. 1B). At the same time, FIGS. 1A to 1B, for explaining a possible exemplary embodiment of a device for determining the proportion of molecular oxygen in respiratory gases.

The device (100) comprises a measurement tube (104) in a metal block (106) with a sound transmitter (102), a sound receiver (102), an infrared transmitter (110) and an infrared receiver (112), an optically pervious crystal window (108), an evaluation unit and two connections for supply and removal of the respiratory gas (114, 116).

The sound transmitter (102) and the sound receiver (102) are piezo oscillators that are used alternately as sound transmitters and sound receivers. These define a sound measurement zone of known length, which preferably extends within or through the measurement tube (104). By means of the sound propagation time measured over the sound measurement zone, the average molar mass of the respiratory gas is determined. The infrared transmitter (110) and infrared receiver (112) define an infrared measurement zone of known length, which preferably passes through the measurement tube (104). The carbon dioxide proportion in the respiratory gas is then determined from the infrared signal received via the infrared measurement system.

The evaluation unit determines the proportion of molecular oxygen in the respiratory gas, the determination taking place by means of formation of the difference between the determined mean molar mass of the respiratory gas and from the determined carbon dioxide proportion of the respiratory gas.

The two connections for supply and/or removal (114, 116) are mounted on mutually opposite ends of the measurement tube (104). By means of the connection for supplying the respiratory gas (114), the latter passes into the device, which it leaves again by means of the connection for removal (116).

### LIST OF REFERENCE CHARACTERS

100 Device according to the invention
102 Sound transmitter and/or sound receiver
104 Measurement tube
106 Metal block
108 Crystal window
110 Infrared transmitter
112 Infrared receiver
114 Connection for supply and removal of the respiratory gas
116 Connection for supply and removal of the respiratory gas

1. Method and device for determining the proportion of molecular oxygen in a respiratory gas, for example in lung function diagnostics, comprising the steps
introduction of the respiratory gas into a measurement tube (104),
transmitting a sound signal by means of a sound transmitter (102) and receiving the sound signal by means of a sound receiver (102)
defining a sound measurement zone by means of the sound transmitter (102) and the sound receiver (102),
determining the average molar mass of the respiratory gas by means of the sound propagation time measured over the sound measurement zone, and
determination of the carbon dioxide proportion of the respiratory gas with a carbon dioxide gas sensor,
characterised in that the determination of the proportion of molecular oxygen in the respiratory gas is performed by means of difference formation from the determined mean molar mass of the respiratory gas and from the determined carbon dioxide proportion.

2. Method according to the preceding claim, characterised in that the determination of the proportion of molecular oxygen (C_{M O}_{₂}) is performed by means of the formula
C_{M O}_{₂}=k₁(M_{respiratory gas}−k₂*C_{M CO}_{₂}−k₃)
where C_{M O}_{₂} is the proportion of molecular oxygen in the respiratory gas M_{respiratory gas} is the average molar mass of the respiratory gas, C_{M CO}_{₂} is the proportion of carbon dioxide in the respiratory gas and k₁, k₂ and k₃ represent dimensional constants.

3. Method according to claim 1, characterised in that the sound measurement zone extends along the flow direction of the respiratory gas in the measurement tube (104) and is passed through a total of two times in mutually opposite directions.

4. Method according to claim 1, characterised in that the sound transmitter (102) is an ultrasound source and/or the sound receiver (102) is an acoustic sensor or a microphone.

5. Method according to claim 1, characterised in that the carbon dioxide gas sensor is an infrared receiver (112), which receives an infrared signal transmitted by an infrared transmitter (110), the frequency of the transmitted infrared signal lying in the absorption spectrum of carbon dioxide and the attenuation of the intensity of the infrared signal representing a measure of the carbon dioxide proportion in the respiratory gas.

6. Method according to the preceding claim, characterised in that an infrared measurement zone is defined by the infrared transmitter (110) and the infrared receiver (112), which crosses the flow direction of the respiratory gas in the measurement tube (104).

7. Method according to claim 1, characterised in that the infrared transmitter (110) is an infrared source and/or the infrared receiver (112) is an optical sensor.

8. Method according to claim 1, characterised in that the temperature and/or the air pressure of the respiratory gas is measured.

9. Device for determining the proportion of molecular oxygen in a respiratory gas according to the method in claim 1, for example in lung function diagnostics, comprising a measurement tube (104), which comprises a sound transmitter (102) and a sound receiver (102), which define a sound measurement zone, in which the average molar mass of the respiratory gas is determined by means of the sound propagation time measured over the sound measurement zone, and a carbon dioxide gas sensor, which determines the carbon dioxide proportion of the respiratory gas,
characterised by an evaluation unit for determination of the proportion of molecular oxygen in the respiratory gas, the evaluation unit determining the difference from the determined average molar mass of the respiratory gas and from the determined carbon dioxide proportion.

10. Device according to claim 9, characterised in that the computation unit determines the difference by means of the following formula:
C_{M O}_{₂}=k₁(M_{respiratory gas}−k₂*C_{M CO}_{₂}−k₃)
where C_{M O}_{₂} is the proportion of molecular oxygen in the respiratory gas M_{respiratory gas} is the average molar mass of the respiratory gas, C_{M CO}_{₂} is the proportion of carbon dioxide in the respiratory gas and k₁, k₂ and k₃ represent dimensional constants.

11. Device according to claim 9, characterised in that the sound transmitter (102) and the sound receiver (102) are arranged such that the sound measurement zone passes along the flow direction of the respiratory gas in the measurement tube (104).

12. Device according to claim 9, characterised in that the sound transmitter (102) is an ultrasound source and/or the sound receiver (102) is an acoustic sensor or a microphone.

13. Device according to claim 9, characterised in that the sound transmitter (102) and the sound receiver (102) are piezo oscillators.

14. Device according to claim 13, characterised in that the piezo oscillators are alternately sound transmitters (102) and the sound receivers (102).

15. Device according to claim 9, characterised in that the carbon dioxide gas sensor is an infrared receiver (112) which receives an infrared signal transmitted by an infrared transmitter (110), the frequency of the transmitted infrared signal lying in the absorption spectrum of carbon dioxide and the attenuation of the infrared signal representing a measure of the carbon dioxide proportion in the respiratory gas.

16. Device according to claim 9, characterised in that the infrared transmitter (110) and the sound receiver (112) are arranged such that they define an infrared measurement zone, which crosses the flow direction of the respiratory gas in the measurement tube (104).

17. Device according to claim 9, characterised in that the infrared transmitter (110) is an infrared source and/or the infrared receiver (112) is an optical sensor.

18. Device according to claim 9, characterised in that the device comprises a means for measuring the temperature and/or the air pressure.

19. Device according to claim 9, characterised in that the device comprises at least one connection for supplying and removing (114) the respiratory gas, said connection being mounted on the measurement tube (104).

20. Device according to claim 19, characterised in that the connection (114) is mounted on the opposite ends of measurement tube (104).
